(19) United States
(12) Patent Application Publication
(10) Pub. No.: US 2025/0185655 A1
(43) Pub. Date: Jun. 12, 2025

(54) PHEROMONE COMPOSITIONS, METHODS OF MAKING, AND THEIR USES

(71) Applicants: Fatma KAPLAN, Woodland, CA (US); Pheronym Inc., Woodland, CA (US)

(72) Inventor: Fatma Kaplan, Woodland, CA (US)

(73) Assignee: Pheronym Inc., Woodland, CA (US)

(21) Appl. No.: 18/686,370

(22) PCT Filed: Sep. 18, 2022
(86) PCT No.: PCT/US2022/043918

§ 371 Date: Feb. 23, 2024

Related U.S. Application Data
(60) Provisional application No. 63/249,114, filed on Sep. 28, 2021.

(51) Int. Cl. A01N 43/16; A01N 63/12; A01P 21/00

(52) U.S. Cl. CPC A01N 43/16; A01N 63/12; A01P 21/00

(57) ABSTRACT

A composition and formulation of pheromone that can be extracted from nematode growth medium and a method of use thereof to induce enhanced plant abiotic stress resistance, and/or promote plant growth, when applied to plants, plant parts, and/or seeds such as by seed treatment, via irrigation water, with additional fertilizers, pesticides, before, during, or after planting are disclosed.

### FIELD OF THE INVENTION

Compositions, methods of making and using said compositions for to enhance abiotic stress resistance in plants/transgenic plants and/or stimulate plant/seed growth.

### BACKGROUND OF THE INVENTION

Abiotic stresses are the primary causes of crop loss worldwide. Abiotic stresses are produced by inappropriate levels of physical components of the environment including temperature extremes (high, low, and freezing temperatures), drought/water deficit, or flooding, microgravity stress, which causes flooding response in plants, lack of oxygen, salinity, low or excess light, UV radiation, oxidative stresses. Even though abiotic stresses cause injury through unique mechanisms that result in specific responses, all forms of abiotic stress seem to elicit a common set of responses. For example, drought/water deficit stress is produced as a secondary stress by chilling, freezing, heat, and salt, as a tertiary stress by radiation, and, of course, as a primary stress during drought/water deficiency. The ability of most organisms to survive and recover from unfavorable conditions is a function of basal and induced tolerance mechanisms.

Induced stress tolerance results from processes involving a number of physiological and biochemical changes, including changes in membrane structure and function, tissue water content, global gene expression, protein, lipid, and primary and secondary metabolite composition. Advances in genome sequencing and global gene expression analysis techniques have further established the multigenic quality of environmental stress responses and the complex nature of temperature.

It is known in the art that nematode pheromone extraction method was developed to stimulate beneficial nematode dispersal behavior to improve beneficial nematodes' efficacy for insect pest control and it was shown that the dispersal behavior was controlled by a mixture of nematode pheromones called ascarosides. However, no other uses are known.

### SUMMARY OF THE INVENTION

In one aspect, this disclosure provides a nematode growth medium extract (dry or liquid) to treat plant seeds or seedlings (transplants) in controlled plant growth environment (e.g., greenhouse, indoor agriculture) application or in the field as seed treatment, root dips for transplants before planting to the field or in irrigation water for specialty crops as annuals (vegetables, ornamentals, etc.) and perennials (trees or fruit and nut orchards, etc.) at planting, post planting or anytime during growth season for plant tolerance to abiotic stress including a drought/water deficit, a low temperature/cold stress, a freezing stress, a high temperature/heat stress, a salt/salinity stress, a low or excessive light, a shading stress, a oxidative stress, an ultraviolet (UV) radiation, a cosmic radiation, a lack of oxygen conditions, a flooding, a heavy metal stress. Methods of manufacture, including purification, storage as dry powder, and use are disclosed for optimal preservation and use of the plant tolerance to abiotic stress.

In an aspect, the present disclosure relates to a formulation comprising:
(a) nematode pheromone including two or more synergistic ascarosides, wherein an ascaroside of the two or more synergistic ascarosides has the formula

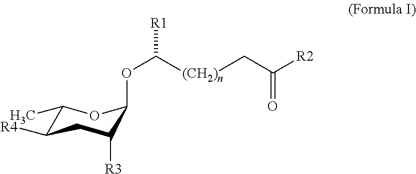

Wherein R1 is H or CH₃,
R2 is OH, CH₃, N-EA, or N-PABA,
R3 is OH, or O-beta-glc,
R4 is OH, O-ICA, O-IAA, or O-ascr, and
n=1, 2, 3, 4, 5, 6, 7, 8, 9, 10, or 11;
and,
(b) one or more agronomically acceptable carrier.

In another aspect, the formulation's said two or more synergistic ascarosides is selected from the group consisting of ascr #1, ascr #2, ascr #3, ascr #4, ascr #5, ascr #6, ascr #6.1, ascr #6.2, ascr #7, ascr #8, ascr #9, ascr #10, ascr #11, ascr #12, icas #9, bhas #18, hbas #3, mbas #3, easc #18, and oscr #9. In particular, it can include ascr #9 and ascr #11.

Yet in an aspect, the formulation can be in any form such as liquid, a lyophilized form, with or without synergistic ascarosides, and have concentrations between from about 0.0625×HCE to about 1×HCE. In the aspect of synergistic ascaroside, it can include up to at least about 45 nmol of ascr #9, and up to at least about 3 nmol of ascr #11.

In one aspect, a method for inducing elevated expression of at least one abiotic resistance traits and/or promoting growth of a plant, plant part, and/or seed is disclosed, which includes: applying a nematode pheromone including one or more ascaroside to said plant, plant part, and/or seed, wherein said one or more ascaroside include the formula I, wherein R1 is H or CH₃, R2 is OH, CH₃, N-EA, or N-PABA, R3 is OH, or O-beta-glc, R4 is OH, O-ICA, O-IAA, or O-ascr, and n=1, 2, 3, 4, 5, 6, 7, 8, 9, 10, or 11; in an effective amount to cause said plant, plant part, and/or seed to exhibit at least one elevated abiotic stress as compared to a control plant and/or seed not so treated. The one or more ascarosides can be selected from the group consisting of ascr #1, ascr #2, ascr #3, ascr #4, ascr #5, ascr #6, ascr #6.1, ascr #6.2, ascr #7, ascr #8, ascr #9, ascr #10, ascr #11, ascr #12, icas #9, bhas #18, hbas #3, mbas #3, easc #18, oscr #9, an/or any combinations thereof. In particular ascr #9 and ascr #11.

In another aspect, the at least one elevated abiotic stress comprises increased tolerance to low temperature/cold stress, freezing stress, high temperature/heat stress, salt/salinity stress, low (shading stress) or excessive light (ultraviolet (UV) radiation and other cosmic radiation), oxidative stress, heavy metal stress, lack of oxygen conditions, flooding, drought/water deficit, microgravity stress, which causes flooding response in plants, and/or any combinations thereof; and/or wherein the application induces growth in said plant parts including plant roots or root hairs, increases plant root hair density, increases length in said plant parts, and/or increase resistance to damage to cold stress and/or drought tolerance for said plant root or said plant parts. In an non-limiting aspect, the tolerance of said plant, plant part, and/or seed to abiotic stresses is improved by at least about: 1%, by 2%, by 5%, by 10%, by 20% by, 30%, by 40%, by 50%, by 60%, by 70%, by 80%, by 90%, or by 100% as compared to plants, plant parts, and/or seed not treated; and/or the nematode pheromone application can prevent damage to roots of said plant and/or plant part from water deficit stress damage by stimulation of growth between about 82% and 92% as compared to plant and/or plant part not under water deficit stress damage.

Yet in another aspect, the application of said nematode pheromone stimulates root formation and dense root hair formation by increases total root length production of up to at least about 128% more roots/seedling and/or at least about up to 122% longer total root length/seedling. The application of said nematode pheromone can result in localized or systemic abiotic stress tolerance throughout said plant and/or plant parts. Alternatively or optionally, the application of said nematode pheromone induces said plant to produce increased amounts of hormones which promote root formation and growth of longer roots, and/or increased production of plant abiotic stress tolerance, wherein said hormones are selected from a group consisting of: cytokinins (CKs), abscisic acid (ABA), gibberellins (GAs), Strigolactone (SL), and combinations thereof.

In another aspect, the application of nematode pheromone increases water deficit, drought tolerance, and/or cold stress tolerance at physiologically relevant pheromone extract concentrations for nematodes.

In one aspect, the said application of said nematode pheromone induce said plant, plant part, and/or seed's abiotic stress tolerance via localized or systemic abiotic stress tolerance throughout said plant, plant parts, and/or seed; wherein said plant part is selected from the group consisting of root, stem, leaf, seed and flower, and wherein said plant is selected from the group consisting of dicots, monocots, annuals, perennials, crop plants, alfalfa, rice, wheat, barley, rye, cotton, sunflower, peanut, corn, oat, millet, flax, potato, sweet potato, bean, green bean, wax bean, lima bean, pea, chicory, lettuce, endive, cabbage, brussel sprout, beet, sugar beet, parsnip, turnip, cauliflower, broccoli, turnip, radish, spinach, onion, garlic, eggplant, pepper, celery, carrot, squash, pumpkin, zucchini, cucumber, melon, yam, carrots, cassava, citrus, strawberry, grape, raspberry, pineapple, soybean, tobacco, tomato, sorghum, sugarcane, ornamental plants, Arabidopsis thaliana, Saintpaulia, petunia, pelargonium, poinsettia, chrysanthemum, carnation, zinnia, poplar, apple, pear, peach, cherry, almond, plum, hazelnuts, banana, apricot, grape, kiwi, mango, melon, papaya, walnut, pistachio, raspberry, blackberry, loganberry, blueberry, cranberry, orange, lemon, grapefruit, tangerine, avocado, and cocoa.

In an aspect, the nematode pheromone can further comprises Bacillus firmus, Bacillus thuringiensis, Bacillus pumilus, Bacillus licheniformis, Bacillus amyloliquefaciens, Bacillus subtilis), Paecilomyces sp., Pasteuria sp., Pseudomonas sp., Brevabacillus sp., Lecanicillium sp., Ampelomyces sp., Pseudozyma sp., Streptomyces sp., Burkholderia sp., Trichoderma sp., Gliocladium sp., avermectin, Myrothecium sp., Paecilomyces spp., Sphingobacterium sp., Arthrobotrys sp., Chlorosplrnium, Neobulgaria, Daldinia, Aspergillus, Chaetomium, Lysobacter spp, Lachnum papyraceum, Verticillium suchlasporium, Arthrobotrys oligospora, Verticillium chlamydosporium, Hirsutella rhossiliensis, Pochonia chlamydosporia, Pleurotus ostreatus, Omphalotus olearius, Lampteromyces japonicas, Brevudimonas sp., Muscodor sp., paraffinic oil, tea tree oil, lemongrass oil, clove oil, cinnamon oil, citrus oil, rosemary oil, pyrethrum, allspice, bergamot, blue gum, camomile, citronella, common jasmine, common juniper, common lavender, common myrrh, field mint, freesia, gray santolina, herb hyssop, holy basil, incense tree, jasmine, lavender, marigold, mint, peppermint, pot marigold, spearmint, ylang-ylang tree, saponins, benzimidazole, a demethylation inhibitor, morpholine, hydroxypyrimidine, anilinopyrimidine, phosphorothiolate, quinone outside inhibitor, quinoline, dicarboximide, carboximide, phenylamide, anilinopyrimidine, phenylpyrrole, aromatic hydrocarbon, cinnamic acid, hydroxyanilide, antibiotic, polyoxin, acylamine, phthalimide, benzenoid, a demethylation, imidazole, piperazine, pyrimidine triazole, myclobutanil, a quinone outside inhibitor, a quinone, a Reynoutria extract, chloronitrile, quinoxaline, sulphamide, phosphonate, phosphite, dithiocarbamate, chloralkythios, phenylpyridin-amine, cyano-acetamide oxime, organophosphates, carbamates, fumigants, avermectin, Myrothecium sp. Bacillus firmus, Pasteuria spp., Paecilomyces, saponins, plant oils, and/or any combinations thereof.

Yet in an aspect, the present disclosure relates to a method to produce ascaroside comprising obtaining a nutrient depleted nematode growth medium selected from the group consisting of liquid broth, agar medium, and insect host cadaver, depleted of nutrients by growing said nematodes to stasis in said growth medium; producing an alcohol-growth medium mixture by adding an alcohol to said growth medium to achieve a final concentration of between about 0% to about 95% of said alcohol in said growth medium; and centrifuging said alcohol-growth medium mixture to remove solid or insoluble matter while maintaining a supernatant from said centrifuging. Alternatively or optionally, the method can further comprise drying the supernatant from said centrifuging to produce a dry extract; resuspending said dry extract in an aqueous medium to produce a water-soluble pheromone extract; centrifuging said water-soluble pheromone extract to remove water-insoluble compounds while maintaining a water-soluble supernatant; and freeze drying or spray drying said water-soluble supernatant to produce a dry plant abiotic stress tolerance inducing composition, wherein said growth medium is selected from the group consisting of a growth medium in which non-pathogenic bacterivore nematodes or insect or entomopathogenic nematodes have been grown. Moreover, the method can further comprise fractionating said supernatant to produce a plurality of ascaroside fractions; and combining different ratios of the plurality of ascaroside fractions.

### BRIEF DESCRIPTION OF THE DRAWINGS

FIG. 1 denotes general structure of ascaroside pheromones.

FIGS. 2A and 2B denote dispersal pheromones structures and their profile during development.

FIGS. 3 (A) and (B) denote dispersal pheromone extracts of the phylogenetically related nematodes species, and for example, in host insect cadaver of Steinernema spp. and Heterorhabditis spp.

FIG. 4 denotes Steinernema carpocapsae pheromone extracts inducing plant root hair and tolerance to water deficit conditions.

FIG. 5 denotes effects of Steinernema feltiae pheromone extracts on wheat seed germination and root hair during cold stress.

FIG. 6 denotes Steinernema feltiae pheromone extracts' effect on plant tolerance to water deficit during cold stress.

FIG. 7 denotes pheromone extract increases the total root number at low temperature during seed germination.

FIG. 8 denotes pheromone extract increases the total root length of seedlings at low temperature at day 12.

FIG. 9 denotes purification procedure of pheromone extract from nematode growth medium.

### DETAILED DESCRIPTION

As used herein, the term “about”, unless context shows otherwise, means a number plus or minus 300% of such number, not including any negative numbers. For example, “about 100 degrees” means temperature from 0 to 300 degrees.

As used herein, the term “derived” means directly isolated or obtained from a particular source or alternatively having identifying characteristics of a substance or organism isolated or obtained from a particular source. In the event that the “source” is an organism, “derived” means that it may be isolated or obtained from the organism itself or from the medium used to culture or grow said organism.

As used herein, the term “HCE” denotes nematode consumed host cadaver equivalent.

It is known in the art that 1 HCE (or 1x HCE) is the physiologically relevant concentration for the nematodes (infective juvenile stage) to exhibit dispersal and infectivity behaviors. 1HCE is typically between about 2 and about 3 mg. It is resuspended in 200 microliter water.

In an embodiment, the present disclosure illustrates new and novel nematode released pheromones' uses that can be derived from the nematode growth medium and induce plant tolerance to abiotic stress including low temperature and water deficient/drought stress, and/or promote root systems, including increasing plant root hair and root numbers and total root length.

The present disclosure also denotes agricultural reamendments with pheromone extracts that can include plant tolerance to drought/water deficient conditions and may be in combination with cold stress and promotes plant root hair density, root length and/or number.

### Composition

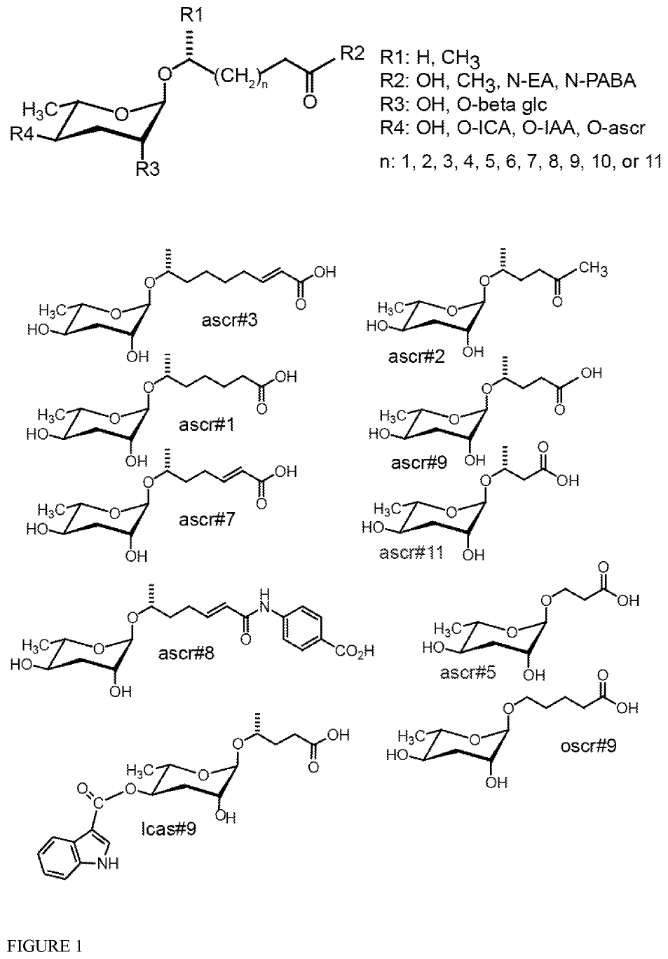

The present disclosure includes, but not limited to a formulation including nematode pheromones, nematode pheromones ascarosides, synergistic ascarosides, naturally and/or chemically derived ascarosides, and compositions forming such formulation. Nematode pheromones ascarosides are a class of compounds such as ascr #1, ascr #2, ascr #3, ascr #4, ascr #5, ascr #6, ascr #6.1, ascr #6.2, ascr #7, ascr #8, ascr #9, ascr #10, ascr #11, ascr #12, icas #9, bhas #18, hbas #3, mbas #3, easc #18, oscr #9, and are known in the art (FIG. 1). They typically compose of a central ascarylose sugar with a variable lipid side chain, and both the lipid chain and ascarylose sugar can have other modifications.

FIG. 1 shows general structure of ascaroside pheromones. N—: N linked; O—: O linked; EA: ethanolamine; PABA: para-amino benzoic acid; ICA: indole carboxylic acid; IAA: indole acetic acid; beta glc: beta glucose; ascr: ascaroside. Examples of ascarosides and their analogs found in nematode growth medium includes asrc #1, ascr #2, ascr #3, ascr #5, ascr #7, ascr #8, ascr #9, ascr #11, oscr #9, and icas #9.

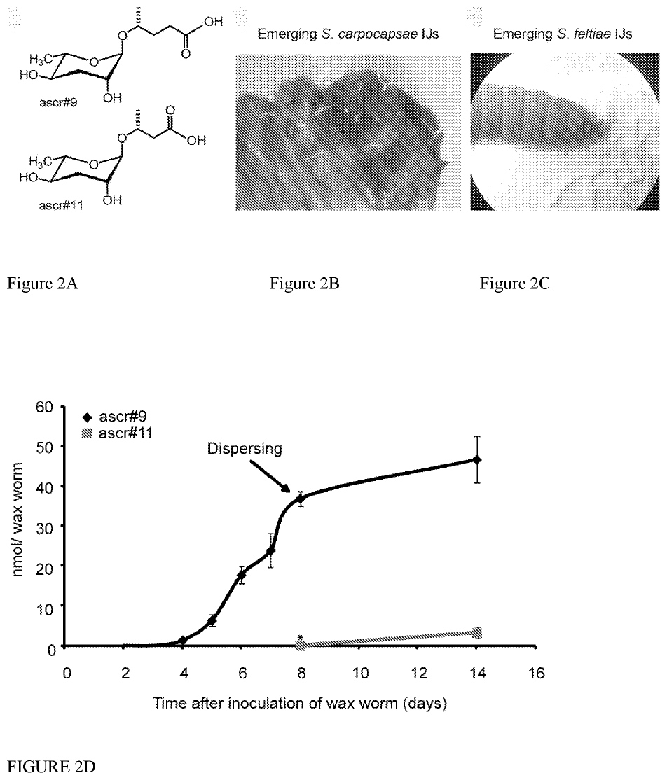

FIG. 2(A) shows structure of ascaroside pheromones found in EPN infected insect host cadavers. Asrc #9 and ascr #11 are identified as part of the pheromone mix responsible for causing the exit of IJs from the consumed insect host cadavers or exodus/dispersal pheromone signal; FIG. 2 (B) denotes consumed insect host cadaver with emerging S. carpocapsae IJs. FIG. 2 (C) denotes consumed insect host cadaver with dispersing S. feltiae IJs. FIG. 2 (D) denotes ascr #9 and ascr #11 profile during S. feltiae development. For each time point, six insect cadavers were analyzed by LC-MS. For the 0-time point, 4 uninfected larvae were analyzed.

The concentration of the pheromones can increase with the nematode development and peaks right before they turn into infective juveniles (FIG. 2) which leave the consumed cadaver.

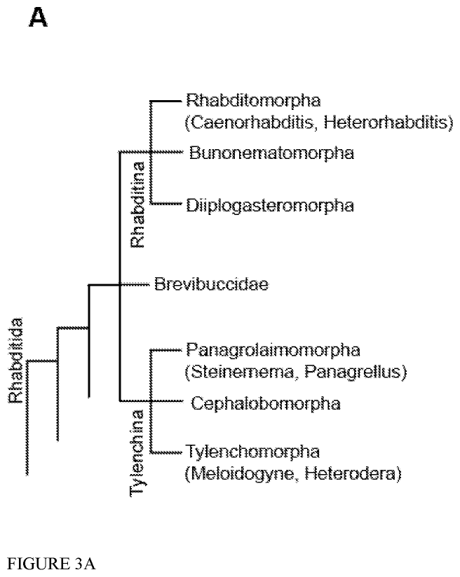

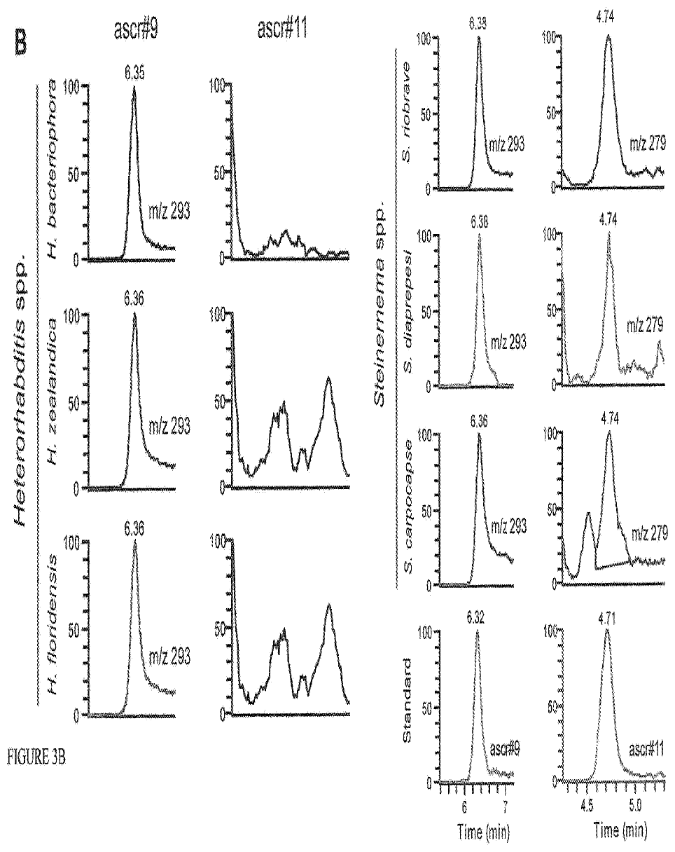

In FIG. 3 (A), a phylogenetic tree for entomopathogenic nematodes, plant parasitic nematodes and C. elegans. The figure is adapted from C. elegans and the biology of nematodes. In FIG. 3 (B), host insect cadaver of Steinernema spp. and Heterorhabditis spp. For each species, four insect (G. mellonella) cadavers infected with both Steinernema spp. or Heterorhabditis spp. were analyzed by LC-MS for ascr #9 and ascr #11 profiles.

Phylogenetically related nematode species, Steinernema and Heterorhabditis species, produce common ascarosides (ascr #11 and ascr #9) which they release to their host cadavers (FIG. 3). See, for example: US20200281213A1, US20210030009A1, U.S. Pat. No. 10,736,326B2, U.S. Pat. No. 8,318,146B1, and/or U.S. Pat. No. 9,534,008B2.

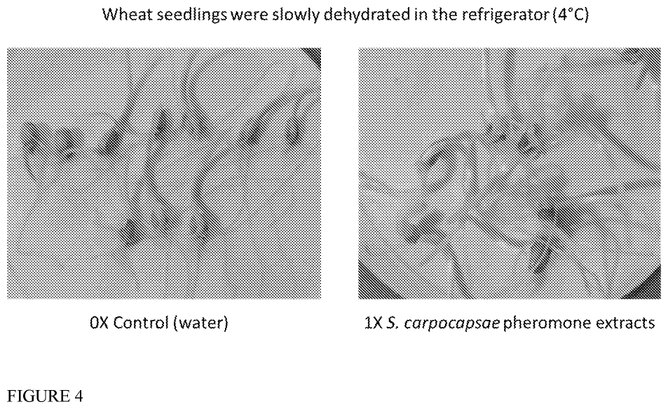

In FIG. 4, 10 wheat seeds treated with water (control) and 10 wheat seeds treated with 1×HCE of S. carpocapsae pheromone extracts for the treatments. Seeds were place at room temperature under normal water conditions for 3 days to germinate and after that both water and pheromone treated seeds were placed in the refrigerator (5° C.) for 17 days and slowly dehydrated.

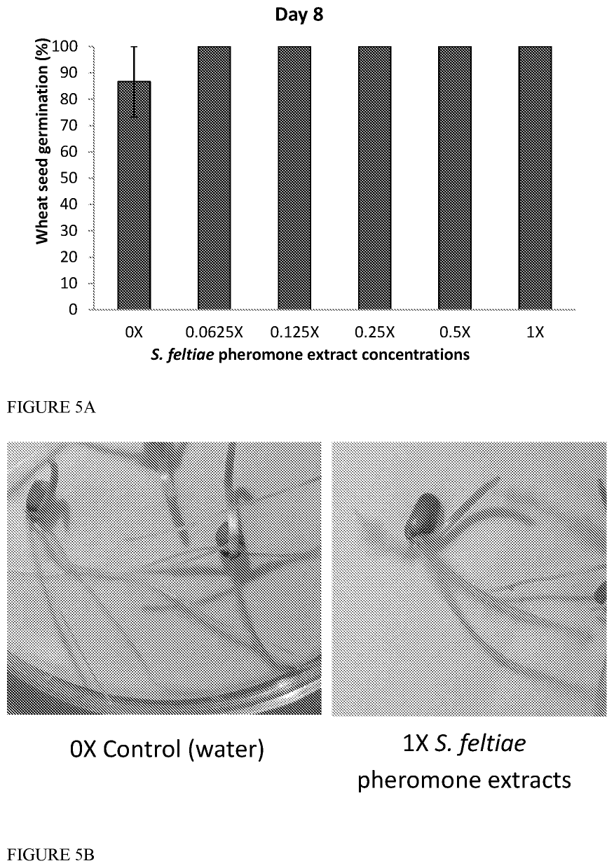

In FIG. 5 (A), wheat seed germination at day 8. Seed were treated with S. feltide pheromone extract with concentrations from 1× (physiologically relevant concentration for nematodes) to 0.0625× and 0×HCE which is the water control. Three experiments with each concentration where each experiment contained five seeds. Total 90 seeds analyzed. Based on student's t-test (p value 0.42), there is no significant effect on seed germination. In FIG. 5 (B), wheat seed root hair density at day 8. Control: 40 ul of water for each seed; Treatment: 40 ul of 1×HCE of S. feltiae pheromone extract for each seed meaning 1 HCE per 10 cm plate moistened with 2 ml of water. Three experiments with each containing 5 seeds for each treatment. Total 30 seeds analyzed. In both A and B, seeds were kept for 3 days at room temperature (RT, 22° C.) and then transferred to 3.5° C. for 5 days.

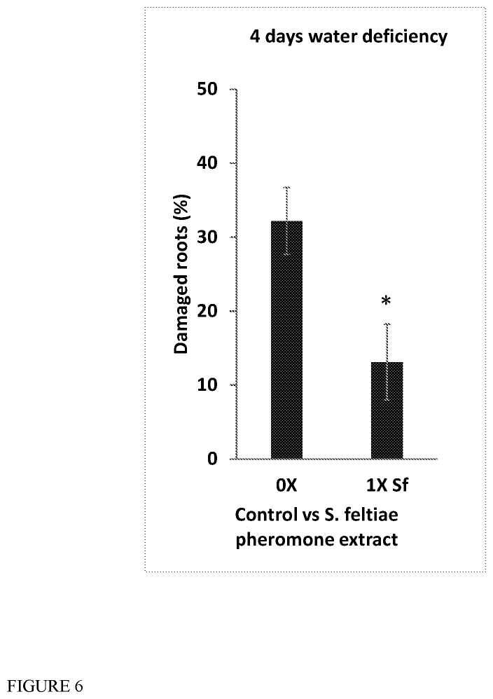

In FIG. 6, Steinernema feltiae pheromone extracts' effect on plant tolerance to water deficit during cold stress. Seeds were either treated with 40 ul of water or 40 ul of 1×HCE pheromone extracts kept 3 days at RT and transferred to 3.5° C.—for 12 days. At day 12, the lids were partially opened to slowly dehydrate each plate at low temperature (3.5° C.). The seedlings were exposed to dehydration for 4 days and analyzed on day 17. The damage to roots was scored as browning roots at the base of the plant root at day 17. Three experiments each containing 5 seedlings. Total 30 seedlings (a total of 128 roots) analyzed. OX: water control; Sf 1×HCE: 40 ul of S. feltiae pheromone extract/per seed, a physiologically relevant concentration for nematodes. The figure data is statistically significant via student's t-test p value 0.005

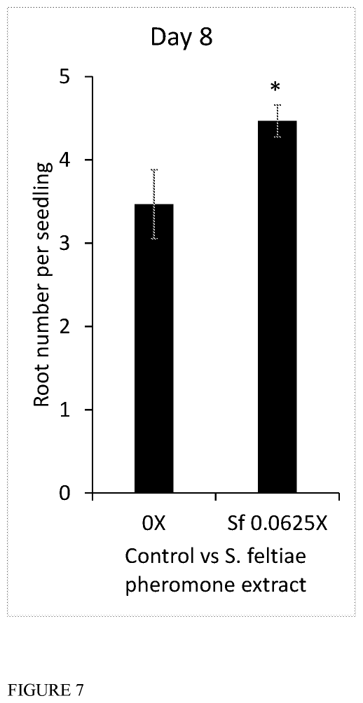

In FIG. 7, it is showed that pheromone extract increases the total root number at low temperature during seed germination. At day 8, root number quantified from three experiments with each containing five seedlings per plate. Total 30 seedlings (total of 119 roots) analyzed. 0×: water control; Sf 0.0625×: S. feltiae pheromone extract. The figure data is statistically significant via student's t-test p value 0.04.

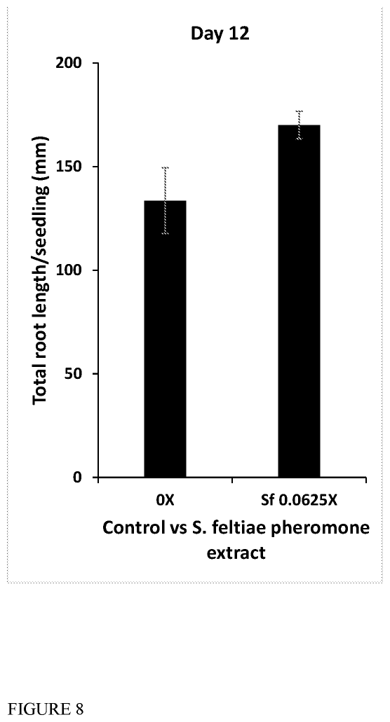

In FIG. 8, three experiments with each experiment per plate contained five seedlings. Total 30 wheat seedlings with a total length of 130 roots quantified at day 12. 0×: water control; Sf 0.0625×: S. feltiae pheromone extract. The figure data is statistically significant via student's t-test p value 0.048

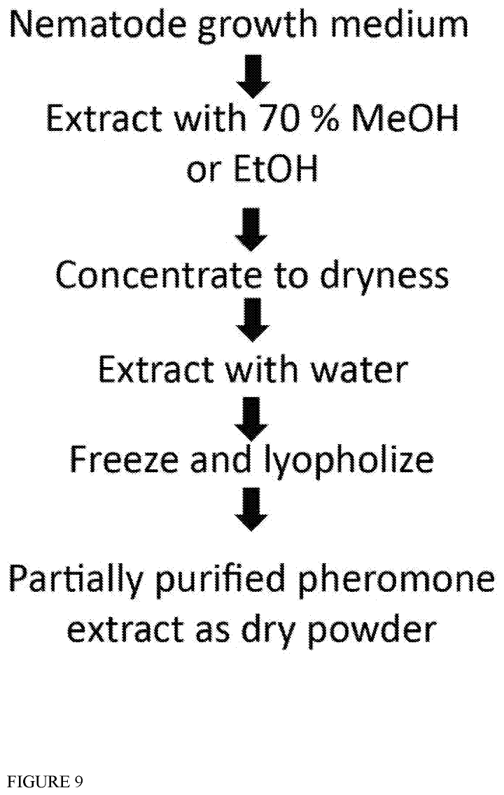

FIG. 9 denotes purification procedure of pheromone extract from nematode growth medium.

### Extraction and Extraction Methods

In an embodiment, the pheromone compositions disclosed can be derived and/or purified from nematode growth medium, including but not limited to, insects, liquid broth, or agar plates. Alternatively, it can be chemically synthesized. Alternatively, additionally, or optionally, the pheromone compositions can be synthesized by modified plant that can produce the compositions, for example, transgenic plants encoding relevant genetic codes to produce such compositions. In some instance, the pheromones, for example, can be extracted using an alcohol, such as but not limited to, 70% methyl alcohol, ethyl alcohol, or combinations thereof, and centrifugation to remove insoluble debris (see FIG. 9).

For example, the pheromone can be extracted with a range of concentration from about 0% to about 95% alcohol. The liquid (supernatant) was removed and concentrated to produce a dry extract by using a stream of nitrogen, by rotary evaporation, or by lyophilization, or by equivalent means. The dry powder was resuspended in water and centrifuged to separate insoluble debris from water-soluble pheromones. The supernatant was concentrated to dryness using a lyophilizer, spray drier or equivalent means for storage. The concentration and ratio of this pheromone mixture was important for the activity on plants. When the extract was diluted up to 16 times (0.0625×HCE) from the physiologically relevant concentration 1×HCE, it increased the root hair density (which was more pronounced at 1× and 0.5× concentrations) and tolerance to drought/water deficient conditions and increased plant root number. When the physiologically relevant concentration was diluted 16 times (0.0625×HCE), it induced plant root length in addition to increased plant root number per seedling. For example, 1 insect host cadaver extract from Galleria mellonella (average weight of G. mellonella larvae, wax worm, is estimated to be 200 microL (microliters) or 232+/−57 mg) is diluted in 200 microL water which is physiologically relevant concentration as 1×HCE and each seed treated with 40 microL of this 1×HCE pheromone extracts on 2 pieces of Whatman filter paper wetted with 2 ml of water in 10 cm petri dishes. This concentration induces plant root hair density and plant drought tolerance. When one insect host cadaver extracts from Galleria mellonella is resuspended in 3200 microL (16 times dilution or 0.0625×HCE) and each seed treated with 40 microL of this dilution on 2 pieces of Whatman filter paper wetted with 2 ml of water in 10 cm petri dishes. The 16 times dilutions induced plant total root length and showed increased number of roots per seedling.

Pheromone extracts of 1×HCE contains up to 45 nmol of ascr #9 and up to 3 nmol of ascr #11 (FIG. 2) when it is extracted either in water or 70% methanol or ethanol can be observed. Both ascr #9 and ascr #11 are found in S. carpocapsae and S. feltiae infected host cadavers (see FIG. 2 and FIG. 3). Furthermore, ascr #9 is found in both Steinernema spp. and Heterorhabditis spp. infected host cadavers (FIG. 3) which were extracted with 70% Methanol.

The extracts are not limited to G. mellonella. Insect host pre-infected weight is considered 1:1 for extract dilution and up to 16 times of the original weight of the extract is active. For the liquid broth, a 1 L (liter) growth medium where the food (as bacteria) density goes down and nematode density goes up and the IJs or analogous life stage (e.g., dauer in C. elegans) forms, the media contains dispersal pheromone which induces plant fitness/tolerance to abiotic stress. One L liquid broth extract (dry powder) is diluted with 1 L water up to 16 L of water. Depending on the goal for drought tolerance (1 L liquid medium dried) the pheromone extracts can be diluted to 1-2 L. For cold tolerance, one L medium (dried) can be diluted up to 16 L. According to one embodiment of the disclosure, seedlings or transplants are treated with resuspended powder for at least about one min (minute), prior to field applications. In alternate embodiments, the resuspended extract is placed in drip irrigation system with or without fertilizers or pesticides and applied to the field. Pheromone extracts can also be applied to seeds as seed treatment or pellets during planting to improve plant stress tolerance to spring freezes or unexpected drought conditions. They are applied to a field with commercially available apparatus.

In one embodiment, effects of nematode pheromone extracts on plant seed germination at a physiologically relevant concentration for the nematodes were evaluated. It did not affect seed germination, but surprisingly, it had the unexpected effect of enhancing plant abiotic stress resistance. The plant roots had more root hair/dense root hair and seemed to tolerate drought/water deficiency during low temperature stress (FIG. 4). This observation was achieved with pheromone extracts from Steinernema carpocapsae infected host cadavers. Other common compounds (or structural analogs) released by EPN species, can be able to see the same and similar response to pheromone extracts from insect infected with other EPN species. Pheromone extracts from S. feltiae infected insect host cadavers were tested. In concentration curve experiments during germination of wheat seeds, we tested nematode pheromone extracts concentrations from 1×HCE (host cadaver equivalent) to 0.0625×HCE. 1×HCE is the physiologically relevant concentration for nematodes. None of the concentrations we tested affect the seed germination (FIG. 5), similar to S. carpocapsae pheromone extracts.

In an embodiment, the present disclosure shows that at physiologically relevant pheromone extract concentrations for nematodes plant root hair density and water deficit/drought tolerance is induced, coupled with cold stress tolerance (FIG. 6). Further, at all concentrations the plant root number was higher in pheromone treated seeds (FIG. 7) with increases at low concentrations. Consistent with the increases in number of roots, the total (combined) root length (FIG. 8) was longer in pheromone extract (low concentration) treated seedlings during cold stress. Methods of making and using the nematode pheromone extract composition according to this disclosure are described in detail herein below and are supported by the Examples provided herein. Purification procedure (FIG. 9): In an embodiment, a ratio of alcohol (EtOH or MeOH) per nematode consumed insect host cadaver was 1:1 (1 ml of 70% EtOH or MeOH per insect cadaver); the effective concentration range was 0% to 95% alcohol. The insect nematode consumed cadavers were homogenized or mixed well with alcohol. Samples were centrifuged and the supernatant was concentrated to dryness via lyophilization or equivalent means. Then samples were extracted with water and centrifuged to separate insoluble compounds. The supernatant was frozen and dried by lyophilization or equivalent means. Previously using LC-MS we showed that these extracts include detectable amounts of nematode pheromones (FIG. 2) such as ascr #9 and ascr #11. Ascr #9 and ascr #11 are common to phyogenetically related entomopathogenic nematode species (FIG. 3). The dry powder was stored either at ambient temperature or in a freezer.

In FIG. 4 and FIG. 5, when the 40 ul of pheromone extract is added to the seeds placed on two Whatman filter papers which were moistened with 2 ml of water. The seeds were imbibed or soaked in water for at least 8 h prior to treatment. In the treatments, the pheromone extracts concentrations ranged from physiologically relevant concentrations (1×) for nematodes to 0.0625× (16 times diluted) concentrations. The physiologically relevant concentration was previously determined as 1 consumed insect host cadaver equivalent (HCE) extracts is resuspended in 200 ul of water. In each experimental plate contained 5 seeds with each treated 40 ul of extracts. So, for 1× pheromone extract concentration, one plate contained only 1 HCE pheromone extract. For 0.0625× pheromone extract concentration treated plates, one plate with 5 seeds contained total 0.0625 HCE per plate.

After addition of the pheromone extracts to the seeds in the 10 cm petri dishes contained two moistened filter papers with 2 ml of MilliQ water, petri dishes were placed in Ziplock bags to keep the moisture for 3 days at room temperature. At day 2, the petri dishes were supplement with 1 ml of water to keep the seeds moistened. At day 3, the seed were transferred to low temperature (3.5° C.). First, the seed germination was monitored to see whether there was any effect on the seed germination. Then we determined other traits for abiotic stress. Seed germination was determined according to ISTA rules. If the wheat seeds had one coleoptile, one radicle and two seminal roots at day 4 and/or day 8, seeds were considered germinated. So, seed germination was not affected at day 4 or day 8 (FIG. 5) at low temperature.

During water deficiency at low temperature (3.5° C.), S. carpocapsae pheromone extracts treated seedling roots in FIG. 4 had lots of root hair and healthy roots. However, the control (water only) seeds were brown and dry. To determine whether this was due common compounds in the nematode growth medium extract including pheromone (FIGS. 2 and 3) we tested a pheromone extract from S. feltiae consumed insect host cadavers from 1× to 0.0625× host cadaver equivalent concentrations. Surprisingly, S. feltiae pheromone extract treated roots with 1× (FIG. 5) and 0.5× concentrations has dense root hairs at day 4 and day 8 like the seeds treated S. carpocapsae pheromone extracts (FIG. 4).

These seedlings with high root density were exposed to water deficit stress for 4 days (FIG. 6), the pheromone treated roots had statistically significantly (students' t-test p<0.005) less damaged roots compare to control.

At low temperature (3.5° C.) seeds had more roots (FIG. 7) and total root length (FIG. 8) when they were treated with pheromone extracts. At low concentration of S. feltiae pheromone extracts from 0.25× to 0.0625×, the seedlings had more roots and total root length. The difference in root numbers were immediately visible at low temperature (3.5° C.) at day 4 and 8 at low temperatures. The total root number was significant at 0.0625× concentration (FIG. 7). When the total root length was evaluated (quantified all the roots and their length together), the seedling treated with 0.0625× pheromone extracts had significantly more total root length (FIG. 8) at day 12.

Dry powder (the nematode pheromone extracts) is resuspended in water and then applied with irrigation water with/without fertilizer or pesticides at planting, after planting during growth season every 2-3 weeks and/or 2 weeks before the drought conditions. It is also applied to seeds as seed treatment or pellets during planting to improve plant stress tolerance to spring freezes or unexpected drought conditions. They are applied to a field with commercially available apparatus.

In an embodiment, the ascaroside composition can, but does not have to be fractionated and differing the ratios of each fraction, then be combined to achieve a purified plant abiotic stress resistance inducing composition.

Non-limiting description of nematodes that can be used to produce pheromone extracts from nematode growth medium using insects or liquid medium in shaker flasks or fermenter, or bioreactors which the present disclosure is applicable include the following commercially available nematodes: Insect nematodes, entomopathogenic nematodes, in the genera Heterorhabditis and Steinernema species such as Steinernema carpocapsae, Steinernema feltiae, Steinernema kraussei, Steinernema glaseri, Steinernema scapterisci, Steinernema riobrave, Steinernema kushidai, Steinernema scarabaei or Heterorhabditis bacteriophora, Heterorhabditis megidis, Heterorhabditis indica, Heterorhabditis marelatus, Heterorhabditis zealandica, Heterorhabditis downesi, Heterorhabditis marelata, Heterorhabditis marelatus.

Insects that are infected by insect nematodes/entomopathogenic nematodes and can be used as growth medium for nematodes include, but are not limited to: Artichoke plume moth, Armyworms, Banana moth, Banana root borer, Billbug, Black cutworm, Black vine weevil, Borers, Cat flea, Chinch bugs, Citrus root weevil, Codling moth, Corn earworm, Corn rootworm, Cranberry girdler, Crane fly, Diaprepes root weevil, Fungus gnats, Grape root borer, Iris borer, Large pine weevil, Leafminers, Mole crickets, Navel orangeworm, Plum curculio, Scarab grubs, Shore flies, Strawberry root weevil, Small hive beetle, Sod webworms Sweetpotato weevil.

Other noncommercial nematodes that produce ascr #9 and/or ascr #11 and their growth medium can be used to produce those pheromones include, but not limited to: Panagrellus redivivus and other Panagrellus spp., Oscheius tipulae, O. carolinencis and Oscheius spp., Caenorhabditis elegans, Caenorhabditis sp.7, Caenorhabditis spp., Rhabditis spp., Pristionchus pacificus and Pristionchus spp., plant parasitic nematodes such as the pinewood nematode, Bursaphelenchus xylophilus, B. mucronatus, Bursaphelenchus spp.

Non-limiting description of other organisms that can be used to produce pheromones ascr #9 and ascr #11: Plants can be fed ascarosides ascr #18 or other long chain ascaroside longer than 5 carbon chain to produce/convert to ascr #9 and/or ascr #11.

For example, plants can convert ascr #18 to ascr #9, ascr #11 (if they are fed/sprayed on aerial plant organs or irrigated on plant roots in the field or in hydroponic plant growth system. In light of the foregoing disclosure, those skilled in the art will appreciate that this disclosure includes a method for obtaining pheromone extracts composition inducing plant tolerance against abiotic stress by an entomopathogenic nematode (“EPN”) dispersal inducing composition by obtaining a nutrient depleted entomopathogenic nematode growth medium selected from liquid broth, agar medium, and insect host cadaver, depleted of nutrients by growing said EPN to stasis in said growth medium. From the growth medium, (e.g. with insect host cadavers, alcohol is added to the cadavers because the volume is very small; with liquid broth, it can be first spray dried or frozen and then lyophilized because the initial volume is large, and then extracted with alcohol), producing an alcohol-growth medium mixture by adding an alcohol to the growth medium to achieve a final concentration of between about 0% to about 95% of the alcohol in the growth medium. The alcohol-growth medium mixture is centrifuged to remove solid or insoluble matter while maintaining a supernatant from the centrifugation step. The supernatant from the centrifuging step can also be dried to produce a dry extract. The dry extract is then resuspended in water or equivalent aqueous medium to produce a water-soluble pheromone extract. The water-soluble pheromone extract is again centrifuged to remove water/aqueous medium insoluble compounds while maintaining a water-soluble supernatant. To preserve the activity, the supernatant from this centrifugation step is dried to produce a dry pheromone extract for plant fitness/tolerance to abiotic stress including drought/water deficit, cold, heat, light stress.

In an embodiment, the alcohol is selected from the ethanol, methanol and mixtures thereof. In another embodiment, the growth medium is selected from a growth medium in which non-pathogenic bacterivore nematodes or insect or entomopathogenic nematodes have been grown.

### Composition Application Methods and Uses

The plant abiotic stress tolerance inducing ascarosides and/or pheromone compositions may be administered to the plant or soil by any techniques known in the art, including, for example, spraying, atomizing, dusting, scattering, coating or pouring. One of skill in the art would be able to determine the appropriate technique for administration according to the specific abiotic stress to be combated, the specific chemical composition and formulation of the compound being employed, the method of applying the compound/formulation, and the locus of treatment.

In one embodiment, the ascarosides and/or pheromone composition may be administered by foliar application. In another embodiment, the compositions may also reach the plants through the root system via the soil (systemic action) by drenching the locus of the plant with a liquid preparation or by incorporating the substances into the soil in solid form, e.g., in the form of granules (soil application). In rice cultivations, these granules may be dispensed over the flooded paddy field.

The compositions of the disclosure may also be applied to tubers or seed grain, for example, by soaking, spraying or drenching the seed grain or tubers in a liquid nematode pheromone and/or ascaroside containing composition or by coating the tubers or seed grain with a solid nematode pheromone (ascaroside) composition.

In this disclosure, pheromone (such as ascarosides) extraction methods and compositions to improve plant root systems and induce plant tolerance to abiotic stress are disclosed herein, including enhanced resistance to: low temperature/cold stress, freezing stress, high temperature/heat stress, salt/salinity stress, low (shading stress) or excessive light (ultraviolet (UV) radiation and other cosmic radiation), oxidative stress, heavy metal stress, lack of oxygen conditions, flooding, drought/water deficit, and/or any combinations thereof.

In an embodiment, the pheromone can be applied to any known transgenic plants already having various traits, such as traits against one or more abiotic stress, one or more herbicides, and/or one or more disease stress.

The pheromone composition can also be applied by coating plant seeds, or germinating seedling roots, before they are planted, or by drenching the roots of existing plants, in situ or in the course of transfer, or by introducing the composition onto the bases of target plants. In an embodiment, the application of the ascaroside composition to target plants induces in plant roots, root hairs, or both, an increased length, number, resistance to damage following stress, an increase in resistance to cold stress, an increase in drought tolerance, and combinations thereof. Application of the ascaroside composition to target plants and/or seeds induces increased tolerance to low temperature/cold stress, freezing stress, high temperature/heat stress, salt/salinity stress, low (shading stress) or excessive light (ultraviolet (UV) radiation and other cosmic radiation), microgravity stress, which causes flooding response in plants, oxidative stress, heavy metal stress, lack of oxygen conditions, flooding, drought/water deficit, and/or any combinations thereof.

The composition according to this disclosure can be used and effective at physiologically relevant pheromone extract concentrations for nematodes, to induce increased plant root hair density and water deficit/drought tolerance, coupled with cold stress tolerance. Moreover, to increase the plant root number, seeds are contacted with the composition. The contacting may result in localized or systemic abiotic stress tolerance throughout the plant, and the part of the plant may be selected from the group consisting of root, stem, leaf, seed, flower, and combinations thereof. The plant can be selected from the group consisting of dicots, monocots, annuals, perennials, crop plants, alfalfa, rice, wheat, barley, rye, cotton, sunflower, peanut, corn, oat, millet, flax, potato, sweet potato, bean, green bean, wax bean, lima bean, pea, chicory, lettuce, endive, cabbage, brussel sprout, beet, sugar beet, parsnip, turnip, cauliflower, broccoli, turnip, radish, spinach, onion, garlic, eggplant, pepper, celery, carrot, squash, pumpkin, zucchini, cucumber, melon, yam, carrots, cassava, citrus, strawberry, grape, raspberry, pineapple, soybean, tobacco, tomato, sorghum, sugarcane, ornamental plants, Arabidopsis thaliana, Saintpaulia, petunia, pelargonium, poinsettia, chrysanthemum, carnation, zinnia, poplar, apple, pear, peach, cherry, almond, plum, hazelnuts, banana, apricot, grape, kiwi, mango, melon, papaya, walnut, pistachio, raspberry, blackberry, loganberry, blueberry, cranberry, orange, lemon, grapefruit, tangerine, avocado, and cocoa.

In another embodiment, the composition disclosed can be applied on various types of cut flowers, roses, grass, turf grass and/or grassland for animals. Non-limiting examples include: Echinochloa phyllopogon, Echinochloa colona, Echinochloa oryzoides, Echinochloa crus-galli, Schoenoplectus spp., Heteranthera spp., Monochoria spp., Bacopa spp., Sagittaria spp., Oryza punctata and Oryza sativa (weedy), Chenopodium sp. (e.g., Chenopodium album, Chenopodium murale), Abutilon sp. (e.g., Abutilon theophrasti), Helianthus sp. (e.g., Helianthus annuus), Ambrosia sp. (e.g., Ambrosia artemesifolia, Ambrosia trifida), Amaranthus sp. (e.g., Amaranthus retroflexus, Amaranthus palmeri, Amaranthus rudis, Amaranthus spinosus, Amaranthus tuberculatus), Convolvulus sp. (e.g., Convolvulus arvensis), Brassica sp. (e.g., Brassica kaber), Taraxacum sp. (e.g., Taraxacum officinale), Solanum sp. (e.g., Solanum nigrum, Solanum elaeagnifolium, Solanum physalifolium, Solanum ptycanthum), Malva sp. (e.g., Malva neglecta), Setaria sp. (e.g., Setaria lutescens), Bromus sp. (e.g., Bromus tectorum, Bromus diandrus, Bromus hordeaceus), Poa sp. (e.g., Poa annua, Poa pratensis), Lolium sp. (e.g., Lolium perenne, Lolium rigidum, Lolium multiflorum L. var. Pace), Festuca sp. (e.g., Festuca arundinaceae, Festuca rubra), Echinochloa sp. (e.g., Echinochloa crus-galli, Echinochloa colona), and particularly, Lambsquarter—Chenopodium album, Redroot Pigweed—Amaranthus retroflexus, Wild Mustard—Brassica kaber, Dandelion—Taraxacum officinale, and Black Nightshade—Solanum nigrum, Oxalis sp. (e.g., Oxalis stricta, Oxalis pes-caprae, Oxalis corniculata); Cyperus sp. (e.g., Cyperus difformis); Conyza sp. (e.g., Conyza canadensis, Conyza sumatrensis, Conyza bonariensis); Sagina sp. (e.g., Sagina procumbens); Veronica sp. (e.g., Veronica hederafolia), Stellaria sp. (e.g., Stellaria media), Rorippa sp. (e.g., Rorippa islandica), Senecio sp. (e.g., Senecio vulgaris), Lamium sp. (e.g., Lamium amplexicaule), Digitaria sp. (e.g., Digitaria sanguinalis, Digitaria ischaemum), Galium sp. (e.g., Galium aparine), Galinsoga sp. (e.g., Galinsoga aristatula), Cardamine sp. (e.g., Cardamine flexuosa, Cardamine hirsuta), Kochia sp. (e.g., Kochia scoparia), Eleusine sp. (e.g., Eleusine indica), Portulaca sp. (e.g., Portulaca oleraceae), Plantago sp. (e.g., Plantago lanceolata), Euphorbia sp. (e.g., Euphornia supina, Euphorbia maculate, Euphorbia esula, Euphorbia prostrata), Erodium sp. (e.g., Erodium cicutarium), Sonchus sp., (e.g., Sonchus oleraceus), Lactuca sp. (e.g., Lactuca serriola), Capsella sp. (e.g., Capsella bursa-pastoris), Leptochloa sp. (e.g., Leptochloa fascicularis, Leptochloa virgata), Raphanus sp. (e.g., Raphanus raphanistrum), Calandrinia sp. (e.g., Calandrinia ciliata), Paspalum sp. (e.g., Paspalum dilatatum), Gnaphalium sp., Cynodon sp. (e.g., Cynodon dactylon, Cynodon hirsutus), Polygonum sp. (e.g., Polygonum arenastrum, Polygonum lapathofolium), Avena fatua, Hordeum sp. (e.g., Hordeum leporinum), Urtica sp. (e.g., Urtica urens), Tribulus terrestris, Sisymbrium sp. (e.g., Sisymbrium irio), Cenchrus sp., Salsola sp. (e.g., Salsola tragus, Salsola kali), Amsinckia sp. (e.g., Amsinckia lycopsoides), Ipomoea sp., Claytonia perfoliata, Polypogon sp. (e.g., Polypogon monspeliensis), Xanthium sp., Hypochaeris radicata, Physalis sp., Eragrostis sp., Verbascum sp., Chamomilla suaveolens, Centaurea sp. (e.g., Centaurea solstitialis), Epilobium brachycarpum, Panicum sp. (e.g., Panicum capilare, Panicum dichotomiflorum), Rumex acetosella, Eclipta sp. (e.g., Eclipta alba, Eclipta prostrata), Ludwigia sp., Urochloa sp. (e.g. Urochloa platyphylla, Urochloa panicoides), Leersia sp., Sesbania sp. (Sesbania herbacea), Rotala sp., Ammania sp., Alternathera philoxeroides, Commelina sp., Sorghum halepense, Parthenium hysterophorus, Chloris truncate, or any combinations thereof.

Various Compositions/Concentrations and their Uses

According to an aspect to this disclosure, the pheromone extract composition can be produced by a method as described herein. Furthermore, further purification, fractions of the composition are produced and combined in differing ratios to produce an active mixture or purified ascaroside pheromones individually or as a mixture In a further embodiment according to the disclosure, the composition according to this disclosure is used to induce tolerance/plant tolerance against abiotic stress including cold shock, drought/water deficit stress in field application by the composition in an aqueous medium applied to drip irrigation water/other means of irrigation, or with pesticide or fertilizer application at planting, post planting during growth season until harvest. Treatment of the plants and soil with the composition described herein may be carried out directly or by allowing the compounds to act on the surroundings, environment, or storage space by the customary treatment methods, for example by immersion, spraying, evaporation, fogging, scattering, painting on and, in the case of propagation material, in particular in the case of seeds, also by applying one or more coats. The composition in an aqueous medium can be applied every 20-30-day intervals to the plant roots in the greenhouse or in the field. Depending on the plant species or plant cultivars, their location and growth conditions (soils, climate, vegetation period, diet), the treatment according to the disclosure may also result in synergistic effects. In one embodiment, combining asc #9 and asc #11 provides synergistic tolerance against abiotic stress and/or increase seedling growth. In another embodiment, for example, reduced application rates and/or a widening of the activity spectrum and/or an increase in the activity of the substances and compositions to be used, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products that exceed the effects which were actually to be expected may occur.

In another embodiment, a stable dry ascarosides composition is provided to induce plant abiotic stress resistance wherein when reconstituted the composition comprises from between about 0.0625×HCE from the physiologic to about 1×HCE. None-limiting example includes that the composition comprises up to about 45 nmol of ascr #9, up to 3 nmol of ascr #11, or both.

In a further embodiment according to this disclosure, it provides a method which include contacting/applying a seed, a plant and/or part of a plant with a composition comprising an effective amount of an isolated ascaroside which increases plant abiotic stress tolerance and promotes plant root system tolerance to one or more abiotic stresses to which the plant is exposed, wherein the ascaroside is selected from the group consisting of ascr #1, ascr #2, ascr #3, ascr #4, ascr #5, ascr #6, ascr #6.1, ascr #6.2, ascr #7, ascr #8, ascr #9, ascr #10, ascr #11, ascr #12, icas #9, bhas #18, hbas #3, mbas #3, easc #18, oscr #9, and combinations thereof.

In certain embodiments according to the disclosure, the method results in increased plant tolerance to abiotic stresses, improving plant root system, and/or preventing plant damage, by at least about: 1%, by 2%, by 5%, by 10%, by 20% by, 30%, by 40%, by 50%, by 60%, by 70%, by 80%, by 90%, or by 100%. In certain embodiments, healthy plant roots are protected from water deficit stress by biostimulation of between about 82% and 92%. In certain embodiments, the pheromone extract treatment stimulates root formation and dense root hair formation and increases total root length as a biostimulant by inducing production of up to at least about 128% more roots/seedling and up to at least about 122% longer total root length/seedling.

Mode of Action for Abiotic Stress Tolerance and/or Growth Promotion

In an embodiment, hormones that induce plant abiotic stress tolerance and root promotion include cytokinins (CKs), abscisic acid (ABA), gibberellins (GAs), Strigolactone (SL). It is known that Cytokinins, Gibberellins, and Strigolactones are involved in lateral root formation, and in promotion of root length.

Yet in another embodiment, the application can result in localized or systemic abiotic stress tolerance throughout said plant and/or plant parts. In one embodiment, the plant is induced to produce increased amounts of hormones which promote root formation and growth of longer roots. In a further embodiment, the contacting results in the plant increasing production of hormones which promote increased root growth and growth of longer roots, and the hormones are selected from a group consisting of: cytokinins (CKs), abscisic acid (ABA), gibberellins (GAs), Strigolactone (SL), and combinations thereof. In yet a further embodiment, the contacting induces increased production of plant abiotic stress tolerance inducing pheromones selected from the group consisting of: cytokinins (CKs), abscisic acid (ABA), gibberellins (GAs), Strigolactone (SL), and/or any combinations thereof.

Additional Agricultural Chemical/Biochemical/Microbe and/or Biostimulants

The composition set forth above may be combined with one or chemical/biochemical/microbe and/or biostimulants with similar and/or different functions, for example with pesticide (e.g., nematicide, fungicide, insecticide), drought tolerance inducer agent, or drought tolerant transgenic plants. The microorganism can include but is not limited to an agent derived from Bacillus sp. (e.g., Bacillus firmus, Bacillus thuringiensis, Bacillus pumilus, Bacillus licheniformis, Bacillus amyloliquefaciens, Bacillus subtilis), Paecilomyces sp. (P. lilacinus), Pasteuria sp. (P. penetrans), Pseudomonas sp., Brevabacillus sp., Lecanicillium sp., Ampelomyces sp., Pseudozyma sp., Streptomyces sp (S. bikinicnsis, S. costaricanus, S. avermitilis), Burkholderia sp., Trichoderma sp., Gliocladium sp., avermectin, Myrothecium sp., Paecilomyces spp., Sphingobacterium sp., Arthrobotrys sp., Chlorosplrnium, Neobulgaria, Daldinia, Aspergillus, Chaetomium, Lysobacter spp, Lachnum papyraceum, Verticillium suchlasporium, Arthrobotrys oligospora, Verticillium chlamydosporium, Hirsutella rhossiliensis, Pochonia chlamydosporia, Pleurotus ostreatus, Omphalotus olearius, Lampteromyces japonicas, Brevudimonas sp., Muscodor sp.

In another embodiments, other agents can also be added such as brassinolide, a natural oil or oil-product having nematicidal, fungicidal insecticidal and/or another agent to increase tolerance drought inducing activity (e.g., paraffinic oil, tea tree oil, lemongrass oil, clove oil, cinnamon oil, citrus oil including but not limited to bitter orange, orange, lemon; rosemary oil, pyrethrum, allspice, bergamot, blue gum, camomile, citronella, common jasmine, common juniper, common lavender, common myrrh, field mint, freesia, gray santolina, herb hyssop, holy basil, incense tree, jasmine, lavender, marigold, mint, peppermint, pot marigold, spearmint, ylang-ylang tree, saponins). Furthermore, the these agents can be a single site anti-fungal agent which may include but is not limited to benzimidazole, a demethylation inhibitor (DMI) (e.g., imidazole, piperazine, pyrimidine, triazole), morpholine, hydroxypyrimidine, anilinopyrimidine, phosphorothiolate, quinone outside inhibitor, quinoline, dicarboximide, carboximide, phenylamide, anilinopyrimidine, phenylpyrrole, aromatic hydrocarbon, cinnamic acid, hydroxyanilide, antibiotic, polyoxin, acylamine, phthalimide, benzenoid (xylylalanine), a demethylation inhibitor selected from the group consisting of imidazole, piperazine, pyrimidine and triazole (e.g., bitertanol, myclobutanil, penconazole, propiconazole, triadimefon, bromuconazole, cyproconazole, diniconazole, fenbuconazole, hexaconazole, tebuconazole, tetraconazole), myclobutanil, and a quinone outside inhibitor (e.g., strobilurin). The strobilurin can include but is not limited to azoxystrobin, kresoxim-methoyl or trifloxystrobin. In yet another particular embodiment, the anti-fungal agent is a quinone, e.g., quinoxyfen (5,7-dichloro-4-quinolyl 4-fluorophenyl ether). The agent may also be derived from a Reynoutria extract.

Furthermore, other ageents can also be a multi-site non-inorganic, chemical fungicide selected from the group consisting of chloronitrile, quinoxaline, sulphamide, phosphonate, phosphite, dithiocarbamate, chloralkythios, phenylpyridin-amine, cyano-acetamide oxime.

In an alternative embodiment, the composition can further include a nematicide. The nematicide can include but is not limited to chemicals such as organophosphates, carbamates, and fumigants, and microbial products such as avermectin, Myrothecium sp. Biome (Bacillus firmus), Pasteuria spp., Paecilomyces, and organic products such as saponins and plant oils.

### Formulation

The pheromone extracts or purified pheromones (ascarosides individually or as a mixture) described herein may be used in unchanged form or together with an agronomically acceptable carrier. The term “agronomically acceptable carrier” includes any carrier suitable for administration to a plant or soil, for example, customary excipients in formulation techniques, such as solutions (e.g., directly sprayable or dilutable solutions), emulsions, (e.g., emulsion concentrates and diluted emulsions), wettable powders, suspensions, soluble powders, powders, dusts, pastes, soluble powders, granules, suspension-emulsion concentrates, encapsulation into polymeric materials, coatable pastes, natural and synthetic materials impregnated with active compound and micro-encapsulations in polymeric substances. These formulations are produced in a known manner, for example by mixing the compounds with agronomically acceptable carrier, such as liquid solvents or solid carriers, optionally with the use of surfactants, including emulsifiers, dispersants, and/foam-formers. If the agronomically acceptable carrier is water, it may also be possible to employ, for example, organic solvents as auxiliary solvents. Suitable liquid solvents include, for example, aromatics (e.g., xylene, toluene and alkylnaphthalenes); chlorinated aromatics or chlorinated aliphatic hydrocarbons (e.g., chlorobenzenes, chloroethylenes and methylene chloride); aliphatic hydrocarbons (e.g., cyclohexane); paraffins (e.g., petroleum fractions, mineral and vegetable oils); alcohols (e.g., butanol or glycol and also their ethers and esters); ketones (e.g., acetone, methyl ethyl ketone, methyl isobutyl ketone and cyclohexanone) and strongly polar solvents (e.g., dimethylformamide and dimethyl sulphoxide). nontoxic carriers be used in the methods of the present disclosure.

Suitable solid agronomically acceptable carriers include, for example, ammonium salts and ground natural minerals (e.g., kaolins, clays, talc, chalk, quartz, attapulgite, montmorillonite and diatomaceous earth); ground synthetic minerals (e.g., highly disperse silica, alumina and silicates); crushed and fractionated natural rocks (e.g., calcite, marble, pumice, sepiolite and dolomite); synthetic granules of inorganic and organic meals; granules of organic material (e.g., sawdust, coconut shells, maize cobs and tobacco stalks). Suitable emulsifiers and foam-formers include, for example, nonionic and anionic emulsifiers (e.g., polyoxyethylene fatty acid esters, polyoxyethylene fatty alcohol ethers, for example, alkylaryl polyglycol ethers, alkylsulphonates, alkyl sulphates and arylsulphonates) protein hydrolysates.

Other materials such as dispersants include, for example, lignin-sulphite waste liquors and methylcellulose. Tackifiers such as carboxymethylcellulose and natural and synthetic polymers in the form of powders, granules or latices, such as gum arabic, polyvinyl alcohol and polyvinyl acetate, as well as natural and non-natural phospholipids, such as cephalins and lecithins, and synthetic phospholipids, can be used in the formulations. Other additives may include, for example, mineral and vegetable oils. Colorants such as inorganic pigments, for example, iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc may also be included in the agronomically acceptable carrier.

For all of the above agronomically acceptable carriers, some are product of nature (such as water), some are not. In one embodiment, the carrier used are not product of nature and are man-made.

The compositions set forth in this disclosure can be formulated in any manner. Non-limiting formulation examples include but are not limited to Emulsifiable concentrates (EC), Wettable powders (WP), Soluble liquids (SL), Aerosols, Ultra-low volume concentrate solutions (ULV), Soluble powders (SP), Microencapsulation, Water dispersed Granules, Flowables (FL), Microemulsions (ME), Nano-emulsions (NE), etc. In any formulation described herein, percent of the active ingredient is within a range of 0.01% to 99.99%.

In another embodiment, the compositions may be in the form of a liquid, gel or solid. A solid composition can be prepared by suspending a solid carrier in a solution of active ingredient(s) and drying the suspension under mild conditions, such as evaporation at room temperature or vacuum evaporation at 65° C. or lower.

Yet in another embodiment, the composition may comprise gel-encapsulated active ingredient(s). Such gel-encapsulated materials can be prepared by mixing a gel-forming agent (e.g., gelatin, cellulose, or lignin) with a culture or suspension of live or inactivated B. megaterium, or a cell-free filtrate or cell fraction of a B. megaterium culture or suspension, or a spray- or freeze-dried culture, cell, or cell fraction or in a solution of pesticidal compounds used in the method of the invention; and inducing gel formation of the agent.

In light of the foregoing disclosure, those skilled in the art will appreciate that the present disclosure includes at least the following embodiments, components, interchangeable elements, and equivalents thereof, including a method for inducing elevated expression of abiotic resistance traits in plants which includes (a) combining at least one nematode ascaroside with a carrier to form an ascaroside composition; and (b) applying the ascaroside composition to a target plant in an amount and for a period sufficient to cause the target plant to exhibit at least one elevated abiotic stress characteristic as compared to a control plant not so treated. From this disclosure, various specific embodiments arise, including wherein the ascaroside composition is produced by the following specific method: a. Obtaining a nutrient depleted nematode growth medium selected from the group consisting of liquid broth, agar medium, and insect host cadaver, depleted of nutrients by growing said nematodes to stasis in said growth medium; b. Producing an alcohol-growth medium mixture by adding an alcohol to said growth medium to achieve a final concentration of between about 0% to about 95% of said alcohol in said growth medium; c. Centrifuging said alcohol-growth medium mixture to remove solid or insoluble matter while maintaining a supernatant from said centrifuging; and d. Drying the supernatant from said centrifuging to produce a dry extract. This extract may be further refined by: e. Resuspending said dry extract in an aqueous medium to produce a water-soluble pheromone extract; f. Centrifuging said water-soluble pheromone extract to remove water-insoluble compounds while maintaining a water-soluble supernatant; and g. Freeze drying or spray drying said water-soluble supernatant to produce a dry plant abiotic stress tolerance inducing composition. The alcohol used can be selected from the group consisting of ethanol, methanol and mixtures thereof. The growth medium van be selected from the group consisting of a growth medium in which non-pathogenic bacterivore nematodes or insect or entomopathogenic nematodes have been grown.

### EXAMPLES

While the foregoing disclosure is considered to provide an adequate written description and enabling disclosure of the invention disclosed and claimed herein, the following examples are provided to ensure that those skilled in the art reading this patent disclosure are put in possession of this invention as of the date of its filing. The specifics of these examples should not, however, be considered as limiting on the scope.

### Example 1

### Pheromone Extracts, Purification and Components of Nematode Pheromone Extracts

Pheromone extracts and purification was conducted as known in art with modifications. In brief, a total of 33 nematode consumed insect host cadavers (G. mellonella larvae) were placed into 70% EtOH and stored at −20° C. until extraction. The insect cadavers were homogenized using 1 g of ceramic zirconium beads (1.25 mm) (ZIRMIL) in 2 ml tubes for 37 sec using a Precellys24 (www.precellys.com) homogenizer. Samples were centrifuged for 15 min at 18400 rcf and the supernatant was lyophilized and resuspended in MILLI-Q water. To facilitate calculations for physiologically relevant concentration of the ascarosides, wax worm volume was estimated at 200 ul; the average weight of wax worms was 232 (+/−57 mg; n=19).

The first reverse-phase solid-phase extraction was performed using Sep-Pak Plus C18 cartridges (Waters corporation, Milford, MA). The initially collected flow through was termed Fraction A. Thereafter, the column was washed with water, collected and saved. Subsequently, the column was eluted with 50% (Fraction B) and 90% MeOH (Fraction C). Individual fractions were analyzed by LC-MS. Fraction A contained ascr #9 and ascr #11. One of the major components, ascr #9, was found to be common in consumed insect host cadavers of Steinernema spp. and Heterorhabditis spp. Ascr #11 was found to be common in the consumed insect host cadavers which were infected with Steinernema spp. (See FIGS. 2 and 3). Briefly, insect hosts (G. mellonella) were infected with H. bacteriophora, H. zealandica, H. floridensis, S. carpocapsae, S. riobrave, or S. diaprepesi. When nematodes began to emerge from insect cadavers, they were placed into 1.5 ml of 70% EtOH and stored at −20° C. until use. Thereafter, insect cadavers were homogenized using 1 g of ceramic zirconium beads (1.25 mm) (ZIRMIL) in 2 ml tubes for 39 sec using a Precellys24 homogenizer. The homogenized cadavers were centrifuged at 3380 rcf for 10 min. The supernatant was diluted with 1 ml of HPLC water and placed at −20° C. and then placed into a speed vac (Speed Vac Plus SC210A, Savant) overnight.

Each cadaver extract was re-suspended in 1 ml of 50% MeOH and centrifuged at 18400 rcf for 15-20 min. Thereafter, samples were diluted in a 1:1 ratio with 0.1% formic acid, yielding sample pH of 4.2. Presence or absence of ascr #9 was determined by LC-MS.

In FIG. 4, FIG. 5, and FIG. 6, it has been demonstrated that with wheat seedlings that both S. carpocapsae and S. feltiae pheromone extracts induced dense root hair and tolerance to low temperature and water deficit. This has not been limited to wheat seedlings, other crops including the group consisting of dicots, monocots, annuals, perennials, crop plants, alfalfa, rice, wheat, barley, rye, cotton, sunflower, peanut, corn, oat, millet, flax, potato, sweet potato, bean, green bean, wax bean, lima bean, pea, chicory, lettuce, endive, cabbage, brussel sprout, beet, sugar beet, parsnip, turnip, cauliflower, broccoli, turnip, radish, spinach, onion, garlic, eggplant, pepper, celery, carrot, squash, pumpkin, zucchini, cucumber, melon, yam, carrots, cassava, citrus, strawberry, grape, raspberry, pineapple, soybean, tobacco, tomato, sorghum, sugarcane, ornamental plants, Arabidopsis thaliana, Saintpaulia, petunia, pelargonium, poinsettia, chrysanthemum, carnation, zinnia, poplar, apple, pear, peach, cherry, almond, plum, hazelnuts, banana, apricot, grape, kiwi, mango, melon, papaya, walnut, pistachio, raspberry, blackberry, loganberry, blueberry, cranberry, orange, lemon, grapefruit, tangerine, avocado, cocoa, and coffee.

### Example 2

Inducing Plant Fitness/Tolerance to Abiotic Stress and/or Improving Root System

Pheromone extracts were extracted from 1000 Galleria mellonella infected with Steinernema spp (S. feltiae or S. carpocapsae) 7 days after nematode emerged out of the consumed cadavers. The pheromones were extracted with 70% Methanol, dried with a rotary evaporator, then extracted with water, and dried. For S. carpocapsae 1 HCE pheromone extracts weight 1.2 mg and for S. feltiae, 1 HCE pheromone extracts weight 3.4 mg.

To induce more roots or total root length at cold temperatures, one HCE pheromone extracts can be diluted up to 3200 ul water (0.0625×HCE) and treat the seeds with 40 ul of the pheromone extracts on a petri dish with 2 filter papers moistened with 2 ml of water. Three days after germination add 1 ml of water and place the plants at cold temperature (3.5° C.). Plants have increased root numbers during germination at day 8 (FIG. 7) and increased total root length at day 12 (FIG. 8). Previously, it was shown that pheromone activity could decrease in 7-10 days at room temperature and 20-30 days at 4° C. The pheromone extract treatment can be repeated at cold temperature monthly or at warm temperature biweekly.

### Example 3

Beneficial Effects of Nematode Pheromone Extract Utilization in Field Application for Improving Plant Fittness/Tolerance to Abiotic Stress and/or as Biostimulants

By using the composition and method according to this disclosure, the plant can increase tolerance to abiotic stresses, improving plant root system and preventing plant damage. The improvements can be by between at least about 1-100%, e.g., by 1%, by 2%, by 5%, by 10%, by 20% by, 30%, by 40%, by 50%, by 60%, by 70%, by 80%, by 90%, or by 100%.

The composition treatment can prevent damage to healthy plant roots from water deficit stress as a biostimulant between 82% and 92% (FIG. 7). The pheromone extract treatment can stimulate root formation and dense root hair formation and increase total root length as a biostimulant 128% more roots/seedling (FIG. 8) and 122% longer total root length/seedling (FIG. 9).

### Example 4

Conditions for Growing Nematodes to Produce Pheromone Extract to Induce Plant Abiotic Stress Tolerance and/or to Improve Plant Root System

Growing nematodes in insects is considered as in vivo growth, growing nematodes outside the insect just with its symbiotic bacteria in liquid or solid media is considered as in vitro growth. Steinernema or Heterorhabditis spp. (Steinernema carpocapsae, Steinernema feltiae, Steinernema kraussei, Steinernema glaseri, Steinernema scapterisci, Steinernema riobrave, Steinernema kushidai, Steinernema scarabaei or Heterorhabditis bacteriophora, Heterorhabditis megidis, Heterorhabditis indica, Heterorhabditis marelatus, Heterorhabditis zealandica, Heterorhabditis downesi, Heterorhabditis marelata) are grown on Galleria mellonella larvae (wax worms, wax moth). The ratio of nematodes is 25-200 IJs per wax worm larvae. Other insect hosts can be used such as Tenebrio molitor (meal worms) larvae navel orangeworm (Ameylois transitella), tobacco budworm (Heliothis virescens), cabbage looper (Trichoplusia ni), pink bollworm (Pectinophora gossypiella), beet armyworm (Spodoptera exigua), corn earworm (Helicoverpa zea), gypsy moth (Lymantria dispar), house cricket (Acheta domesticus) and various beetles (Coleoptera). After two days, the infected larvae are placed into new 6 cm diameter petri dishes and the white trap method is used to collect IJs. It takes about 7-10 days from infection to emergence of IJs. Once IJs form and leave the cadavers (or 3 days after emergence of the IJs), cadavers are collected to extract pheromones.

An average of pheromone extracts from S. feltiae from one insect host cadaver weighs 3.4 mg that can be diluted from 0.2 ml to 3.2 ml and per seed we would use 0.04 ml from either of the dilution. (So, from 1000 grubs, we would produce 3400 mg of pheromone extracts in 0.2 L to 3.2 L). For the seeds, 0.04 ml can be added to induce abiotic stress tolerance and increased root hair density and more root formation.

Alternatively, nematode IJs are introduced to a pure culture of their symbiont in a nutritive medium at optimum growth temperature in a solid agar medium or a liquid culture with aeration in shake flasks, stirred bioreactors, airlift bioreactors. Media for in vitro approaches can be animal product based (e.g., pork kidney or chicken offal) or includes various ingredients such as peptone, yeast extract, eggs, soy flour, and lard. Exemplary in vitro medium recipes for solid or liquid fermentation are known in the art. In vitro growth recipes are the same for both liquid and solid medium except for the agar.

The liquid medium does not contain agar, solid medium does because agar is the solidifying agent. For Liver-kidney Agar (for 500 ml): Beef liver (50 g), Beef kidney (50 g), Sodium chloride 2.5 g (0.5% final concentration), Agar, 7.5 g (1.5% agar, final concentration), 500 ml distilled H₂O. For Lipid Agar (for 1 L): Nutrient broth (8 g), Yeast extract (5 g), Magnesium chloride hexahydrate 10 ml (0.2 g/ml), Corn oil, 4 ml, Corn syrup, 96 ml combine 7 ml corn syrup in 89 ml heated H₂O and swirl for homogeneity, Agar (15 g), Distilled H₂O (890 ml).

Nematode IJs are inoculated into liquid medium with a density between about 300-4,000 nematodes per ml at 25 or 28 degrees centigrade until nematodes reproduce and form IJs again (about 20-60% newly formed IJs). Such cultures may be synchronized cultures or unsynchronized cultures. Once new IJs are formed in the liquid cultures, nematodes are separated from the liquid medium. Then medium is centrifuged to remove the bacteria. The supernatant is spray dried or frozen and lyophilized. The dry medium is extracted with alcohol and dried. Then it is extracted with water and dried. If the starting volume of the medium is 1 L, the dry extract is resuspended with 1-2 L and can be diluted up to 16 L of water. The liquid pheromone suspension can be applied with drip irrigation in controlled environment, vertical farming, greenhouses or in the field in the drip irrigation system with/without fertilizers or pesticides at planting or after planting seeds, seedlings, transplants or trees.

What is claimed is:

1. A formulation comprising:
(a) nematode pheromone including two or more synergistic ascarosides, wherein an ascaroside of the two or more synergistic ascarosides has the formula 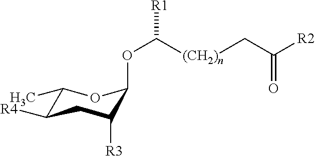
Wherein R1 is H or CH₃,
R2 is OH, CH₃, N-EA, or N-PABA,
R3 is OH, or O-beta-glc,
R4 is OH, O-ICA, O-IAA, or O-ascr, and
n=1, 2, 3, 4, 5, 6, 7, 8, 9, 10, or 11;
and,
(b) one or more agronomically acceptable carrier.

2. The formulation according to claim 1, wherein said two or more synergistic ascarosides is selected from the group consisting of ascr #1, ascr #2, ascr #3, ascr #4, ascr #5, ascr #6, ascr #6.1, ascr #6.2, ascr #7, ascr #8, ascr #9, ascr #10, ascr #11, ascr #12, icas #9, bhas #18, hbas #3, mbas #3, easc #18, and oscr #9.

3. The formulation according to claim 2, wherein the two or more synergistic ascaroside comprises ascr #9 and ascr #11.

4. The formulation according to claim 3, wherein the formulation is in a lyophilized form including synergistic ascarosides concentration between from about 0.0625×HCE to about 1×HCE.

5. The formulation according to claim 3, wherein the two or more synergistic ascaroside comprises up to at least about 45 nmol of ascr #9, and up to at least about 3 nmol of ascr #11.

6. A method for inducing elevated expression of at least one abiotic resistance traits, and/or promoting growth of a plant, plant part, and/or seed, the method comprising:
applying a nematode pheromone including one or more ascaroside to said plant, plant part, and/or seed, in an effective amount to cause said plant, plant part, and/or seed to tolerate at least one elevated abiotic stress, and/or promoting growth of said plant, plant part, and/or seed, as compared to a plant, plant part, and/or seed not so treated,
wherein said one or more ascaroside include the formula 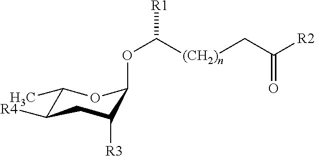
Wherein R1 is H or CH₃,
R2 is OH, CH₃, N-EA, or N-PABA,
R3 is OH, or O-beta-glc,
R4 is OH, O-ICA, O-IAA, or O-ascr, and
n=1, 2, 3, 4, 5, 6, 7, 8, 9, 10, or 11.

7. The method according to claim 6, wherein said one or more ascarosides is selected from the group consisting of ascr #1, ascr #2, ascr #3, ascr #4, ascr #5, ascr #6, ascr #6.1, ascr #6.2, ascr #7, ascr #8, ascr #9, ascr #10, ascr #11, ascr #12, icas #9, bhas #18, hbas #3, mbas #3, easc #18, and oscr #9.

8. The method according to claim 7, wherein the one or more ascaroside comprises ascr #9 and ascr #11.

9. The method according to claim 7, wherein the at least one elevated abiotic stress comprises increased tolerance to low temperature/cold stress, freezing stress, high temperature/heat stress, salt/salinity stress, low (shading stress) or excessive light (ultraviolet (UV) radiation and other cosmic radiation), oxidative stress, heavy metal stress, lack of oxygen conditions, flooding, drought/water deficit, microgravity stress, which causes flooding response in plants, and/or any combinations thereof.

10. The method according to claim 6 wherein the application of the nematode pheromone induces growth in said plant parts including plant roots or root hairs, increases plant root hair density, increases length in said plant parts, and/or increase resistance to damage to cold stress and/or drought tolerance for said plant root or said plant parts.

11. The method according to claim 6, wherein said tolerance of said plant, plant part, and/or seed to said abiotic stresses is improved by at least about: 1%, by 2%, by 5%, by 10%, by 20% by, 30%, by 40%, by 50%, by 60%, by 70%, by 80%, by 90%, or by 100% as compared to plants, plant parts, and/or seed not treated.

12. The method according to claim 10, wherein said nematode pheromone application prevents damage to roots of said plant and/or plant part from water deficit stress damage by stimulation of growth between about 82% and 92%.

13. The method according to claim 10, wherein the application of said nematode pheromone stimulates root formation and dense root hair formation by increases total root length production of said plant and/or seed of up to at least about 128% more roots/seedling and/or at least about up to 122% longer total root length/seedling.

14. The method according to claim 6, wherein the application of said nematode pheromone results in localized or systemic abiotic stress tolerance throughout said plant and/or plant parts.

15. The method according to claim 6, wherein the application of said nematode pheromone induces said plant to produce increased amounts of hormones which promote root formation and growth of longer roots, wherein said hormones are selected from a group consisting of: cytokinins (CKs), abscisic acid (ABA), gibberellins (GAs), Strigolactone (SL), and combinations thereof.

16. The method according to claim 6, wherein said nematode pheromone application induces increased production of plant abiotic stress tolerance inducing pheromones selected from the group consisting of: cytokinins (CKs), abscisic acid (ABA), gibberellins (GAs), Strigolactone (SL), and combinations thereof.

17. The method according to claim 6, wherein the application of the nematode pheromone increases water deficit, drought tolerance, and/or cold stress tolerance at physiologically relevant pheromone extract concentrations for nematodes.

18. The method according to claim 6, wherein the said application of said nematode pheromone induce said plant, plant part, and/or seed's abiotic stress tolerance via localized or systemic abiotic stress tolerance throughout said plant, plant parts, and/or seed; wherein said plant part is selected from the group consisting of root, stem, leaf, seed and flower, and wherein said plant is selected from the group consisting of dicots, monocots, turf grass, annuals, perennials, crop plants, alfalfa, rice, wheat, barley, rye, cotton, sunflower, peanut, corn, oat, millet, flax, potato, sweet potato, bean, green bean, wax bean, lima bean, pea, chicory, lettuce, endive, cabbage, brussel sprout, beet, sugar beet, parsnip, turnip, cauliflower, broccoli, turnip, radish, spinach, onion, garlic, eggplant, pepper, celery, carrot, squash, pumpkin, zucchini, cucumber, melon, yam, carrots, cassava, citrus, strawberry, grape, raspberry, pineapple, soybean, tobacco, tomato, sorghum, sugarcane, ornamental plants, Arabidopsis thaliana, Saintpaulia, petunia, pelargonium, poinsettia, chrysanthemum, carnation, zinnia, poplar, apple, pear, peach, cherry, almond, plum, hazelnuts, banana, apricot, grape, kiwi, mango, melon, papaya, walnut, pistachio, raspberry, blackberry, loganberry, blueberry, cranberry, orange, lemon, grapefruit, tangerine, avocado, and cocoa.

19. The method according to claim 6, wherein said nematode pheromone further comprises brassinolide, Bacillus firmus, Bacillus thuringiensis, Bacillus pumilus, Bacillus licheniformis, Bacillus amyloliquefaciens, Bacillus subtilis), Paecilomyces sp., Pasteuria sp., Pseudomonas sp., Brevabacillus sp., Lecanicillium sp., Ampelomyces sp., Pseudozyma sp., Streptomyces sp., Burkholderia sp., Trichoderma sp., Gliocladium sp., avermectin, Myrothecium sp., Paecilomyces spp., Sphingobacterium sp., Arthrobotrys sp., Chlorosplrnium, Neobulgaria, Daldinia, Aspergillus, Chaetomium, Lysobacter spp, Lachnum papyraceum, Verticillium suchlasporium, Arthrobotrys oligospora, Verticillium chlamydosporium, Hirsutella rhossiliensis, Pochonia chlamydosporia, Pleurotus ostreatus, Omphalotus olearius, Lampteromyces japonicas, Brevudimonas sp., Muscodor sp., paraffinic oil, tea tree oil, lemongrass oil, clove oil, cinnamon oil, citrus oil, rosemary oil, pyrethrum, allspice, bergamot, blue gum, camomile, citronella, common jasmine, common juniper, common lavender, common myrrh, field mint, freesia, gray santolina, herb hyssop, holy basil, incense tree, jasmine, lavender, marigold, mint, peppermint, pot marigold, spearmint, ylang-ylang tree, saponins, benzimidazole, a demethylation inhibitor, morpholine, hydroxypyrimidine, anilinopyrimidine, phosphorothiolate, quinone outside inhibitor, quinoline, dicarboximide, carboximide, phenylamide, anilinopyrimidine, phenylpyrrole, aromatic hydrocarbon, cinnamic acid, hydroxyanilide, antibiotic, polyoxin, acylamine, phthalimide, benzenoid, a demethylation, imidazole, piperazine, pyrimidine triazole, myclobutanil, a quinone outside inhibitor, a quinone, a Reynoutria extract, chloronitrile, quinoxaline, sulphamide, phosphonate, phosphite, dithiocarbamate, chloralkythios, phenylpyridin-amine, cyano-acetamide oxime, organophosphates, carbamates, fumigants, avermectin, Myrothecium sp. Bacillus firmus, Pasteuria spp., Paecilomyces, saponins, plant oils, and/or any combinations thereof.

20. A method to produce ascaroside comprising the steps of:
obtaining a nutrient depleted nematode growth medium selected from the group consisting of liquid broth, agar medium, insect host cadaver depleted of nutrients by growing said nematodes to stasis in said growth medium, and a growth medium in which non-pathogenic bacterivore nematodes and insect or entomopathogenic nematodes have been grown;
producing an alcohol-growth medium mixture by adding an alcohol to said growth medium to achieve a final concentration of between about 0% to about 95% of said alcohol in said growth medium;
centrifuging said alcohol-growth medium mixture to remove solid or insoluble matter while maintaining a supernatant from said centrifuging;
drying the supernatant from said centrifuging to produce a dry extract;
resuspending said dry extract in an aqueous medium to produce a water-soluble pheromone extract;
centrifuging said water-soluble pheromone extract to remove water-insoluble compounds while maintaining a water-soluble supernatant; and
freeze drying or spray drying said water-soluble supernatant to produce a dry plant abiotic stress tolerance inducing composition;
fractionating said supernatant to produce a plurality of ascaroside fractions; and
combining different ratios of the plurality of ascaroside fractions.

21-23. (canceled)
